(19) United States
(12) Patent Application Publication
(10) Pub. No.: US 2007/0134788 A1
(43) Pub. Date: Jun. 14, 2007

(54) Manipulation of chemical and/or biological species using magnetic field modulators

(75) Inventors: Karl Schumacher, Singapore (SG); Jackie Y. Ying, Singapore (SG); Jeremy Ming Hock Loh, Singapore (SG); Shyi Herng Kan, Singapore (SG)

Correspondence Address: WOLF GREENFIELD & SACKS, P.C., 600 ATLANTIC AVENUE, BOSTON, MA, 02210-2206, US

(73) Assignee: Agency for Science, Technology and Research, Centros (SG)

(21) Appl. No.: 11/297,797

(22) Filed: Dec. 8, 2005

(51) Int. Cl. C12N 5/06; C12M 3/00

(52) U.S. Cl. 435/325; 435/289.1

(57) ABSTRACT

The present invention relates to methods and apparatus for manipulating chemical and/or biological species (e.g., cells) and, more specifically, to methods and apparatus for manipulating chemical and/or biological species using magnetic fields. In one embodiment, a method of manipulating cells involves the patterning of cells using a magnetic field modulator, which can modulate the magnetic field created by a magnetic field source. The cells may be magnetically susceptible in some cases; for instance, they may be tagged with magnetic particles. When a fluid containing cells is brought in contact with a surface, and the magnetic field modulator is positioned proximate the surface, the cells may form a pattern proximate the surface by aligning with portions of the modulated magnetic field. The position of the pattern of cells may be at least partially determined by the position of the magnetic field modulator in relation to the surface. Advantageously, in certain embodiments, the magnetic field modulator is not integrally connected to the surface. Thus, patterns of cells can be formed on various types of surfaces and the magnetic field modulator may be repositioned without altering the surface. In one particular embodiment, moving the magnetic field modulator from a first to a second position enables the formation of a second pattern of cells on the surface. This method can allow the formation of patterns comprising multiple cell types on a single surface. The methods and apparatuses of the present invention can be used in a variety of settings. One such setting involves the patterning of multiple cell types proximate a surface, i.e., for studying cell-cell interactions. Another setting involves the formation of three-dimensional cellular structures (e.g., tissues).

### FIELD OF INVENTION

The present invention relates to methods and apparatus for manipulating chemical and/or biological species and, more specifically, to methods and apparatus for manipulating chemical and/or biological species using magnetic fields.

### BACKGROUND

The ability to manipulate chemical species (e.g., chemical reagents) or biological species (e.g., cellular material, polymers, proteins, DNA, and the like) on a microscale is important in many applications. Such applications are in the fields of tissue engineering, biotechnology, microanalysis, and microsynthesis, amongst others. Depending on the application, the manipulations may involve positioning (e.g., patterning), separating, and/or transporting the species.

One approach to manipulating species involves the use of magnetic-based systems. Often, the targeted species is associated with a magnetic material (e.g., by tagging the species with magnetic beads) and the species can be attracted or separated using magnetic fields. In many cases, the species are attracted towards magnetic regions patterned on a substrate. Subsequently, the species may form patterns on the substrate defined by these magnetic regions. U.S. Patent Publication No. 2003/0022370, published Jan. 30, 2003, entitled “Magnet Immobilization of Cells,” by Casagrande et al., gives one example of this approach. This publication involves immobilizing one or more cells associated with a magnetic material on a substrate on which are located one more magnetic receptacle(s). Alternatively, the device arrays cells associated with a magnetic material on a substrate having a pattern of magnetic receptacles disposed thereon. The localized magnetic field gradient may be derived from permanent magnets embedded in the substrate.

While many approaches enable positioning of chemical and/or biological species through the use of magnetic fields, these techniques typically require magnetic components that are fabricated in, or on, a substrate. Advances in the field that could, for instance, enable positioning of species in defined spatial arrangements independent of the substrate would find application in a number of different fields.

### SUMMARY OF THE INVENTION

Methods and apparatus associated with manipulating chemical and/or biological species are provided.

In one embodiment, a method of forming a pattern of cells proximate a surface is provided. The method comprises providing a surface for directing formation of a pattern of cells, providing a magnetic field, positioning a magnetic field modulator in a first position in relation to the surface and modulating the magnetic field, wherein the magnetic field modulator is not integrally connected to the surface, contacting the surface with a fluid containing a first set of cells, and forming a first pattern of cells proximate the surface, wherein the position of the first pattern of cells proximate the surface is determined at least partially by the first position of the magnetic field modulator.

In another embodiment, a method of forming a pattern of cells proximate a surface, is provided. The method comprises providing a surface for directing formation of patterns of cells, providing a magnetic field, positioning a magnetic field modulator in a first position in relation to the surface and modulating the magnetic field, forming a first pattern of a first set of cells proximate the surface, wherein the position of the first pattern proximate the surface is determined at least partially by the first position of the magnetic field modulator, positioning the magnetic field modulator in a second position in relation to the surface and modulating the magnetic field, wherein the first and second positions are different, and forming a second pattern of a second set of cells proximate the first pattern of the first set of cells, wherein the position of the second pattern is determined at least partially by the second position of the magnetic field modulator.

In another embodiment, a method of forming a pattern of cells proximate a surface is provided. The method comprises providing a surface for directing formation of a pattern of cells, providing a magnetic field, positioning a magnetic field modulator in a first position relative to the surface and modulating the magnetic field, contacting the surface with a fluid containing a first set of cells, forming a first pattern of cells proximate the surface, wherein the position of the first pattern is determined at least partially by the first position of the magnetic field modulator, and causing the cells to form a three-dimensional cellular structure.

In another embodiment, an apparatus for forming a pattern of cells proximate a surface is provided. The apparatus comprises a surface for forming a pattern of cells, a magnetic field, and a magnetic field modulator positioned adjacent the magnetic field and the surface, wherein the magnetic field modulator is not integrally connected to the surface.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1G show a scheme illustrating a method of forming one or more patterns of cells proximate a surface according to one embodiment of the invention;

FIGS. 2A and 2B show schematic illustrations of a magnetic field modulator according to another embodiment of the invention;

FIGS. 3A and 3B show simulations of magnetic flux lines and magnetic field intensity, respectively, induced by a magnetic field modulator according to another embodiment of the invention;

FIG. 4 shows an example of an experimental setup for patterning cells using a magnetic field modulator according to another embodiment of the invention;

FIG. 5 is a photograph of a pattern of cells formed proximate a surface using a magnetic field modulator according to another embodiment of the invention;

FIG. 6 shows a series of induced magnetic fields simulated for different magnetic field modulator designs according to another embodiment of the invention;

FIG. 7 is a photograph of another pattern of cells formed proximate a surface using magnetic field modulators according to another embodiment of the invention;

FIG. 8 is a magnified photograph of a pattern of cells formed proximate a surface using a magnetic field modulator according to another embodiment of the invention;

FIGS. 9A-9D are photographs of patterns of different cell types formed on surfaces using magnetic field modulators according to another embodiment of the invention;

FIGS. 10A-10B are photographs of primary cells deposited on surfaces without the use of a magnetic field modulator;

FIGS. 10C-10F are photographs of primary cells patterned on surfaces using magnetic field modulators according to another embodiment of the invention;

FIG. 11 is a photograph showing the formation of thin columns of cells proximate a surface using a magnetic field modulator according to another embodiment of the invention;

FIGS. 12A-12C are photographs showing the patterning of multiple cell types proximate a surface using a magnetic field modulator according to another embodiment of the invention;

FIG. 13 shows a schematic illustration of another magnetic field modulator according to another embodiment of the invention;

FIGS. 14A-14C show schematic illustrations of cell patterns that can be formed using the magnetic field modulator shown in FIG. 13 according to another embodiment of the invention;

FIGS. 15A-15C show schematic illustrations of another magnetic field modulator according to another embodiment of the invention;

FIGS. 16A and 16B show photographs of tubular cell patterns formed using the magnetic field modulator shown in FIG. 15 according to another embodiment of the invention;

FIG. 17A shows a schematic diagram illustrating a method of tagging cells with magnetic particles according to another embodiment of the invention;

FIG. 17B-17E are photographs of cells tagged with magnetic particles according to another embodiment of the invention;

FIG. 18 is a photograph of an example of an experimental setup for patterning cells using a magnetic field modulator according to another embodiment of the invention; and

FIG. 19 is another photograph of an example of an experimental setup for patterning cells using a magnetic field modulator according to another embodiment of the invention.

### DETAILED DESCRIPTION

The present invention relates to methods and apparatus for manipulating chemical and/or biological species (e.g., cells) and, more specifically, to methods and apparatus for manipulating chemical and/or biological species using magnetic fields. In one embodiment, a method of manipulating cells involves the patterning of cells using a magnetic field modulator, which can modulate the magnetic field created by a magnetic field source. The cells may be magnetically susceptible in some cases; for instance, they may be tagged with magnetic particles. When a fluid containing cells is brought in contact with a surface, and the magnetic field modulator is positioned proximate the surface, the cells may form a pattern proximate the surface by aligning with portions of the modulated magnetic field. The position of the pattern of cells may be at least partially determined by the position of the magnetic field modulator in relation to the surface. Advantageously, in certain embodiments, the magnetic field modulator is not integrally connected to the surface. Thus, patterns of cells can be formed on various types of surfaces and the magnetic field modulator may be repositioned without altering the surface. In one particular embodiment, moving the magnetic field modulator from a first to a second position enables the formation of a second pattern of cells on the surface and/or on the first patterns of cells (or otherwise in relation to the first pattern of cells, whether contacting the first pattern of cells, the surface, both, or another entity). This method can allow the formation of cell patterns comprising multiple cell types on a single surface.

The methods and apparatuses of the present invention can be used in a variety of settings. One such setting involves the patterning of multiple cell types proximate a surface, i.e., for studying cell-cell interactions. Another setting involves the formation of three-dimensional cellular structures (e.g., tissues).

Although the primary description below involves patterning of cells proximate surfaces, it is to be understood that the invention can be used to manipulate other chemical and/or biological species by way of positioning, separating, and/or transporting the species in other settings.

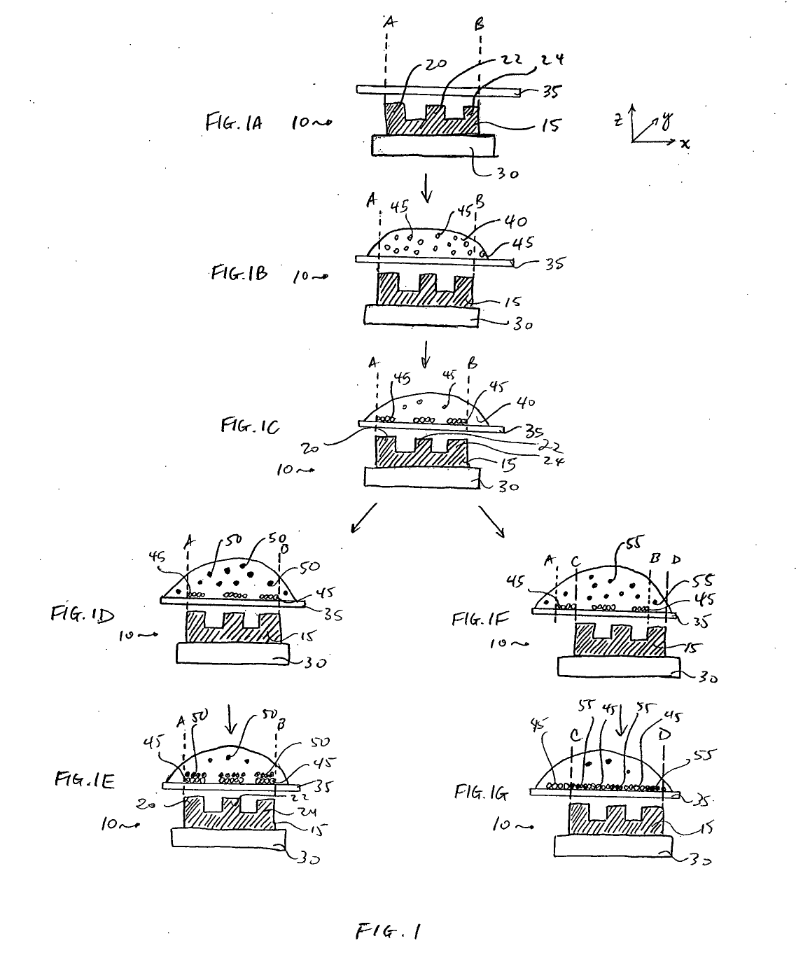

FIG. 1 illustrates a method of forming a pattern of cells according to certain embodiments of the present invention. In the embodiment illustrated in FIG. 1A, apparatus 10 includes magnetic field modulator 15, including protrusions 20, 22, and 24, positioned proximate magnetic field source 30. Magnetic field source 30 may be any object that emits a magnetic field, such as a permanent magnet. Magnetic field modulator 15 can modulate the magnetic field emitted from magnetic field source 30 to produce highly localized magnetic field gradients (i.e., modulated magnetic fields). In some cases, modulation of a magnetic field can be controlled by the shape and spacing of the protrusions of the modulator, as discussed in more detail below. In the arrangement illustrated, highly localized magnetic fields will be directed in approximate alignment with protrusions 20-24. Those of ordinary skill in the art will readily be able to select and/or construct different modulators for establishment of a wide variety of magnetic field patterns useful in connection with the invention.

Magnetic field modulator 15 and magnetic field source 30 may be placed proximate surface 35. In the embodiment shown in FIG. 1A, magnetic field modulator 15 is not in physical contact with surface 35. In other embodiments, however, magnetic field modulator 15 may be in direct physical contact with surface 35, or may be separated from surface 35 by one or more additional materials, as described below. In some cases, the position of the magnetic field modulator in relation to the surface, i.e., in the x, y, or z directions, can influence the formation of a pattern of cells proximate the surface. The horizontal (x) position of magnetic field modulator 15 in relation to surface 35 may be defined, for instance, by positions A and B.

As shown in FIGS. 1B and 1C, fluid 40 containing a plurality of cells 45 may be deposited on top of surface 35. In some instances, the cells may be magnetically susceptible, e.g., they may be tagged with magnetic particles, as described in more detail below. The highly localized magnetic field gradients produced by the protrusions of magnetic field modulator 15 can cause cells 45 to align with the magnetic field gradient. For instance, as illustrated in FIG. 1C, cells 45 align with protrusions 20, 22, and 24, i.e., where the magnetic field gradients are the highest. Thus, patterns of cells can be formed actively on surface 35 by magnetic attraction. The position of the pattern of cells on surface 35 may be at least partially determined by the position of magnetic field modulator 15 in relation to surface 35. For instance, the placement of magnetic field modulator between positions A and B can cause, in some instances, the formation of a pattern of cells between positions A and B.

Certain methods of the invention can allow the patterning of multiple cell types on or in relation to a single surface. For instance, as shown in FIGS. 1D and 1E, a second set of cells defined by cells 50 can be deposited on surface 35. Cells 50 may also be magnetically susceptible and may align with the high magnetic field gradients produced by protrusions 20, 22 and 24 of modulator 15. Cells 50 can form a second pattern of cells, which may, for instance, align on top of the first pattern of cells formed by cells 45.

As illustrated in the embodiments shown in FIG. 1, magnetic field modulator 15 is not integrally connected to surface 35. Advantageously, this allows the magnetic field modulator to be repositioned without altering the surface. For instance, as shown in FIG. 1F, after the patterning of a first pattern of cells defined by cells 45 on surface 35, modulator 15 may be repositioned in relation to surface 35. E.g., modulator 15 may be moved from positions A and B to positions C and D. Of course, in other embodiments, the surface can be moved while the magnetic field modulator stays stationary, or, both the surface and the magnetic field modulator can be repositioned if desired. Depositing a second set of cells, defined by cells 55, on surface 35 can allow the formation of a second pattern of cells whose position is at least partially determined by the new position of modulator 15, e.g., positions C and D. As shown in FIG. 1G, the second pattern of cells can be positioned between the first pattern of cells, thereby forming alternating columns of cells 45 and 55. In certain embodiments, especially ones in which magnetic field modulator 15 is not integrally connected to surface 35, patterns of cells can be formed on various types of surfaces 35, as discussed in more detail below.

In the description herein concerning the formation of patterns of cells proximate a surface using a magnetic field modulator, those of ordinary skill in the art can select surfaces, types and shapes of materials, arrangement of components, etc. based upon general knowledge of the art and available reference materials concerning preferential magnetic attraction between certain materials, in combination with the description herein.

### EXAMPLE 1

Magnetic Field Modulator

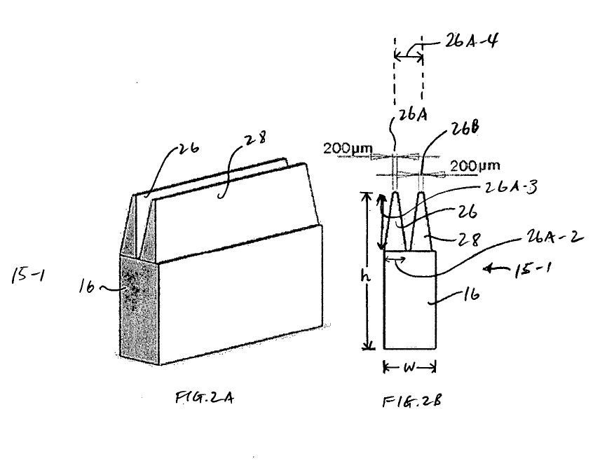

FIGS. 2A and 2B show exemplary illustrations of a magnetic field modulator according to one embodiment. Magnetic field modulator 15-1 can include a base portion 16 and protrusions 26 and 28. As discussed in more detail below, the shape of protrusions 26 and 28 can influence the modulation of a magnetic field gradient. For instance, in some cases, widths 26A and 28A of protrusions 26 and 28, respectively, define the resolution of the features in a pattern of cells formed proximate a surface. In other cases, however, the resolution of features of a cell pattern can also be influenced by the position of the magnetic field modulator in relation to the surface, as discussed below. In one particular embodiment, magnetic field modulator 15-1 was fabricated with width, w=5 mm, and height, h=8 mm. Each protrusion of the modulator was 200 μm wide at the tip (26A and 26B), 1 mm wide at the base (26A-2), 3 mm in height (26A-3=hₒ), and spaced 500 μm apart from the adjacent protrusion (26A-4). Of course, appropriate shapes, dimensions, configurations, and materials used to form magnetic field modulators can vary and may be determined by those of ordinary skill in the art using routine experimentation.

When a magnetic field modulator is placed proximate a magnetic field produced by a magnetic field source (e.g., a permanent magnet), the magnetic field modulator can manipulate the magnetic flux lines and magnetic field intensity of the magnetic field, and form a modulated magnetic field with a magnetization M. M is a measure of the magnetic moment per unit volume of material. It can also be expressed in per unit mass, or by the specific magnetization (μₒ). The magnetic field of the source material is called the applied field (H), and is the total field that would be present if the field were applied to a vacuum. The magnetic induction (B) is the total flux of magnetic field lines through the cross-sectional area of the magnetic field modulator. Considering both lines of force from the applied field and the magnetization of the magnetic field modulator, the magnetic induction is B=μₒ(H+M).

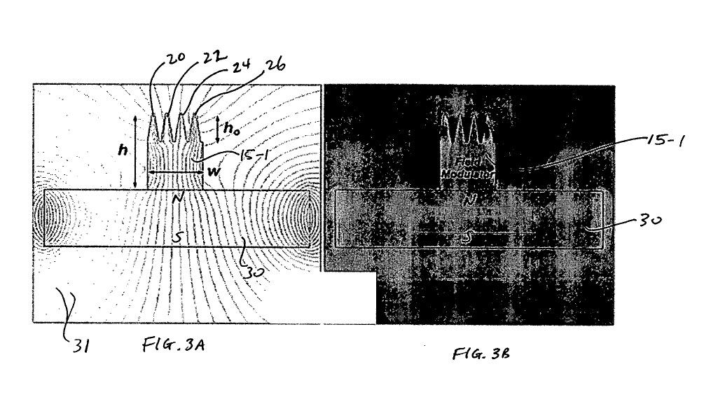

FIGS. 3A and 3B show simulations of the magnetic flux lines and magnetic field intensities, respectively, of magnetic source 30 when magnetic field modulator 15-1 is positioned proximate magnetic source 30. Magnetic source 30 has its north pole oriented vertically upwards, with its flux lines 31 extending outwards from the center of the magnet. The magnetostatic simulations can be performed using ANSOFT Maxwell®2D software. The software created the required finite element mesh automatically for calculating the desired magnetic field solution for analysis and manipulation. Magnetic field modulator 15-1 was a three-dimensional object, but the software analyzed a two-dimensional cross-section of the model, and then generated a solution for that cross-section using finite element analysis. After the first run, the initial mesh was refined to increase the accuracy of the solution with a targeted minimal energy error (usually below 0.8%). A total of 11000 mesh triangles were used to refine the areas around the field modulator and its background.

As shown in FIG. 3A, the shape of the magnetic flux line pattern around magnetic field modulator 15-1 was influenced by protrusions 20, 22, 24, and 26, which were in the shape of saw-teeth. The field lines that were close to each other represented a strong magnetic force; lines that were spread out represented a comparatively weaker magnetic force. The protrusions created high magnetic field gradients (i.e., magnetic fields which change in strength in a certain given direction) near the tip of the protrusions. The magnetic field strength was simulated as shown in FIG. 3B, and the magnetic induction, which was a measure of the magnetic field induced at the tip of the protrusions, was estimated. For the two outer protrusions (protrusions 20 and 26) of magnetic field modulator 15-1, the magnetic induction was simulated to be 0.4337 T, which is represented by a darker shade in FIG. 3B. A lower field concentration (0.3224 T) was measured for the two inner protrusions (protrusions 22 and 24) of the modulator.

### EXAMPLE 2

Experimental Setup for Patterning Cells

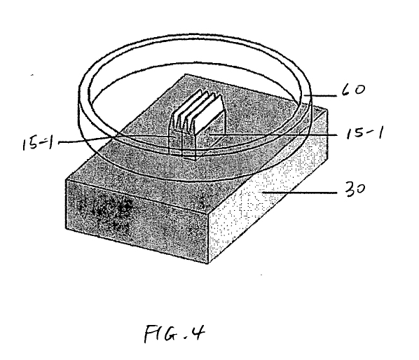

FIG. 4 shows an example of an experimental setup for patterning cells according to one embodiment of the invention. As illustrated in this figure, multiple magnetic field modulators 15-1 can be positioned adjacent one another to form a larger modulator for patterning cells. A surface, such as that of petri dish 60, or a glass slide, can be positioned proximate (i.e., on top of) magnetic field modulators 15. Cells in a petri dish can, be attracted by magnetic force onto the bottom of petri dish 60 under sterile conditions.

### EXAMPLE 3

Patterning Cells Proximate a Surface

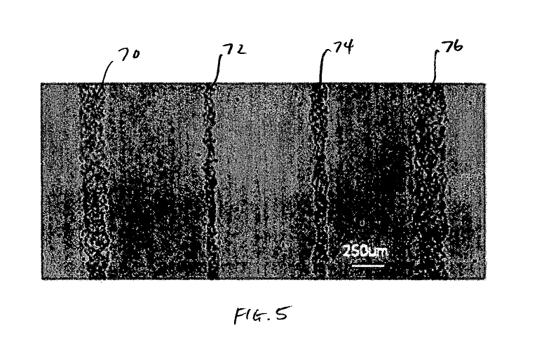

In one particular embodiment, magnetic field modulator 15-1 was used to pattern Madin-Darby canine kidney (MDCK) epithelial cells proximate a surface. A NdFeB permanent magnet with an internal magnetization of ˜1 T was used as the magnetic field source. The MDCK epithelial cells, which were tagged with streptavidin-conjugated superparamagnetic beads (Dynal Biotech) of 1 μm-diameter, were patterned proximate a surface (e.g., the surface of a sterilized petri dish or a glass slide). The surface and cells were positioned proximate the magnetic field modulator and magnetic field source, e.g., as show in FIG. 4. Within minutes, fine lines of cells could be observed near the protrusions of magnetic field modulator 15-1. FIG. 5 shows a pattern of cells in the form of columns after 3 hours of incubating the cells. The inner pair of cell columns (columns 72 and 74) was measured having an average width of approximately 80 μm; these cells aligned with protrusions 22 and 24 of magnetic field modulator 15-1, respectively. The two outer cell columns (columns 70 and 76) were measured having an average width of approximately 250 μm; these cells aligned with protrusions 20 and 26 of magnetic field modulator 15-1, respectively. These experimental findings substantiated the simulation results, demonstrating that the dimensions of the patterned cells can be controlled by the shape and dimensions of the protrusions of the magnetic field modulator. In particular, since the induced field concentrations at the two outer protrusions were higher than those of the inner protrusions, more cells were attracted towards the outer regions to form wider columns at the outer regions (columns 70 and 76);

As demonstrated above, the design of magnetic field modulator 15-1 induced a non-homogeneous field across the four protrusions, resulting in non-uniform cell patterns, i.e., cell columns of substantially different widths, as shown in FIG. 5. In order to generate uniform cell patterns, i.e., cell columns of substantially similar widths, the magnetic field modulator can be designed to have protrusions of different dimensions and/or spacing-between protrusions.

### EXAMPLE 4

Controlling Modulation of a Magnetic Field

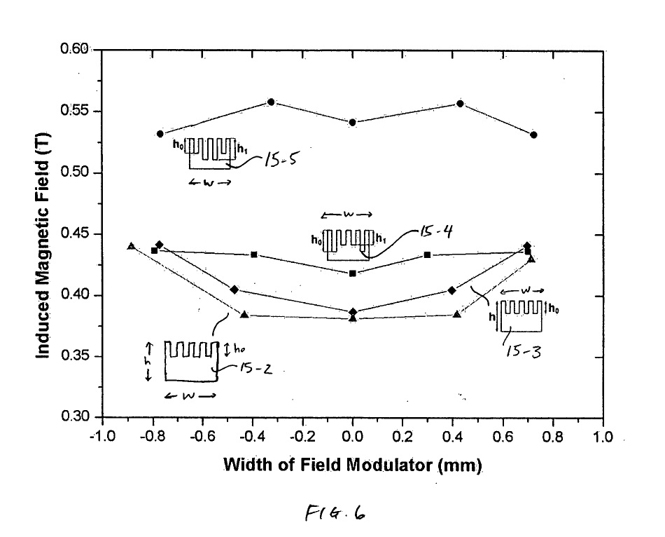

The shape and configuration of a magnetic field modulator can influence modulation of a magnetic field. To demonstrate the effect of dimensions of a magnetic field modulator on the modulated fields produced by the modulator, a series of magnetic field modulators 15-2, 15-3, 15-4, and 15-5 were designed with different dimensions, and the modulated magnetic fields produced at the center of each protrusion were simulated for each modulator (FIG. 6). Magnetic field modulators 15-2, 15-3, 15-4, and 15-5 were designed to have a total width (w) of 1.8 mm, including five protrusions of vertical sidewalls 200 μm-wide, spaced 200 μm apart from adjacent protrusions. Magnetic field modulator 15-2 had an overall height (h) of 3 mm and a protrusion height (h₀) of 0.4 mm. Induced fields of 0.38 T and 0.44 T were estimated for the three inner teeth and the two outer teeth, respectively, of modulator 15-2. By reducing h to 2 mm and maintaining h₀ at 0.4 mm in the modulator design for magnetic field modulator 15-3, an overall increase in the field strength was achieved compared to magnetic field modulator 15-2. Thus, the strength of a modulated magnetic field can be varied by varying the height of the mnagnetic field modulator. The strength of the modulated magnetic field can also be controlled, in some cases, by the distance of the modulator from the surface and/or by the distance of the modulator from the magnetic field source. In general, magnetic field strength decreases with increasing distance from the magnetic field source.

In the illustration described above, manipulating h did not produce a uniform induced field for all five protrusions across modulator 15-3. To increase the field strength of the three inner protrusions to the level of the two outer protrusions, a staggered protrusion height design can be adopted, whereby h₀ and h₁ designated the heights of the two outer protrusions and the three inner protrusions, respectively. For instance, increasing h₀ to 0.7 mm while keeping h₁ at 0.4 mm led to a more substantially uniform field across the protrusions in magnetic field modulator 15-4, except for the middle protrusion (FIG. 6). To increase the magnetic field strength for the middle protrusion, magnetic field modulator 15-5 was designed to have h₀=0.4 mm and h₁=0.7 mm. This design led to increases in the induced fields for all five protrusions to a substantially similar level. The staggered height approach, coupled with a reduction of the overall modulator height h to 1 mm, provided a 20% increase in the induced fields to 0.55±0.012 T for the 5 protrusions, compared to magnetic field modulator 15-2. This example shows that the strength of a modulated magnetic field can be controlled by the relative dimensions of the magnetic field modulator.

### EXAMPLE 5

Aligning Cells Proximate a Surface

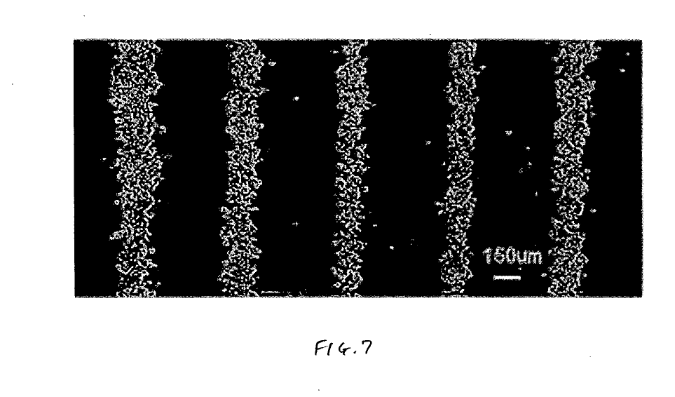

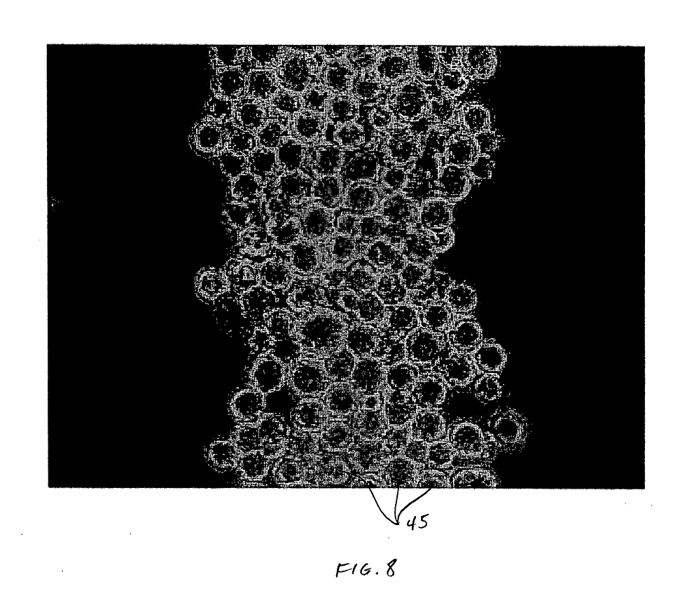

Magnetic field modulator 15-5 was used to modulate the magnetic field of a permanent magnet and to align cells according to this modulated magnetic field. As shown in FIG. 7, MDCK cells were patterned into five columns of substantially uniform width of approximately 175 μm using magnetic field modulator 15-5. The cells in each of the columns aligned over the protrusions of magnetic field modulator 15-5. The width of the-columns of cells were controlled at least in part by the shape and dimensions (e.g., height and width) of the protrusions. FIG. 8 shows a close-up photograph of cells patterned in a column. As shown in FIG. 8, cells can be packed closely and can attach to the surface within four hours of exposure to a magnetic field.

### EXAMPLE 6

Patterning of Different Cell Types

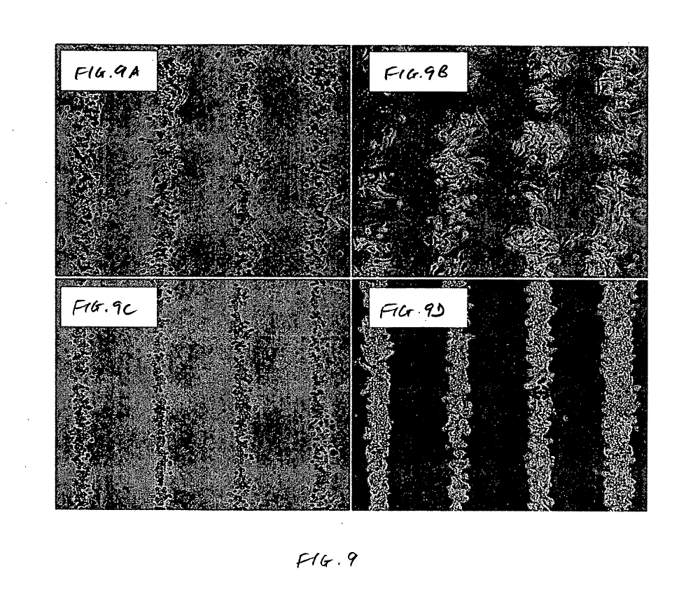

FIGS. 9A-9D show that embodiments of the invention can be used to pattern several different cell types. FIG. 9A shows the formation of neuronal cell patterns; FIG. 9B shows patterning of muscle cells; FIG. 9C shows patterning of fibroblasts; and FIG. 9D shows patterning of epithelial cells. Of course, other types of cells can also be patterned.

### EXAMPLE 7

Patterning of Primary Cells Proximate a Surface

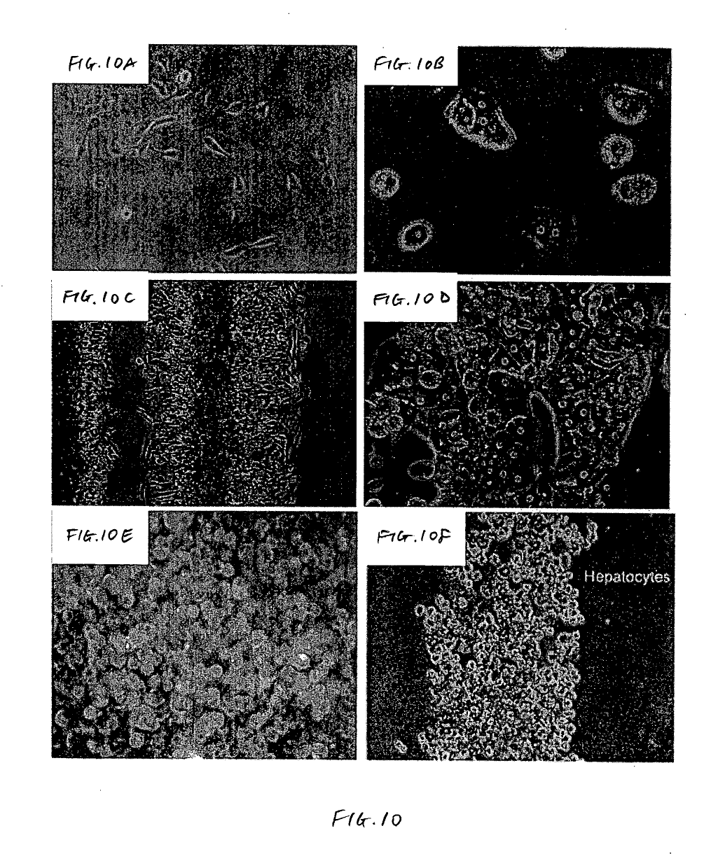

Culturing of primary cells is, in some cases, more challenging than that of other cells lines (e.g., secondary and immortalized cells). FIG. 10 shows that embodiments of the invention can be used to pattern primary cells proximate a surface. Growth of cells patterned proximate a surface can be controlled such that a monolayer of cells, or multi-layers of cells, are formed proximate the surface. FIGS. 10A and 10B show primary human proximal tubule cells and primary rat hepatocytes, respectively, cultured on a surface without the use of magnetic field modulators. Culturing of these cells without magnetic field modulators lead to randomly distributed cell patterns. In some embodiments, a magnetic field modulator can be used to form dense patterns of cells, as demonstrated in FIGS. 10C and 10D. FIGS. 10C and 10D show primary human proximal tubule cells and primary rat hepatocytes, respectively, cultured proximate a surface using a magnetic field modulator. FIG. 10E shows primary hepatocytes labeled with wheat germ agglutinin-FITC. FIG. 10F shows a photograph of a dense, aligned pattern of hepatocytes formed using a magnetic field modulator. Normal cell shapes were observed with these cells, indicating that changes in the cell phenotype after magnetic micro-patterning did not occur.

### EXAMPLE 8

Controlling Dimensions of Cell Pattern Features

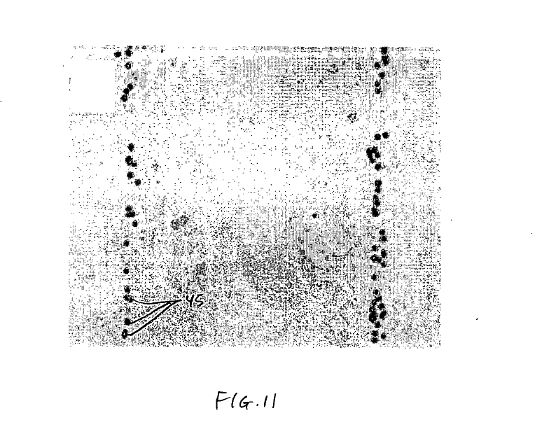

In some instances, dimensions of the features (e.g., columns) of a cell pattern can be controlled at least in part by the density of cells deposited proximate a surface. For instance, in one embodiment, a high density of cells (e.g., on the order of 10⁵ cells/mL or greater) can produce columns of packed cells approximately 200 μm wide. In another embodiment, a low density of cells (e.g., on the order of 500 to 1000 cells/mL) can produce columns of cells on the order of a few cells wide (e.g., approximately 8-20 μm wide); as shown in FIG. 11. In some cases, single columns or other patterns of cells can be patterned proximate a surface, and the dimensions of the patterns may be less than 10 μm.

Dimensions of cell pattern features can also be controlled by the position of the magnetic field modulator in relation to the surface. For instance, in some cases, a magnetic field modular positioned in physical contact with the surface can allow the formation of strong magnetic field intensities near the surface. This can allow the formation of relatively wide patterns of cells proximate a surface. E.g., in one embodiment, the width of a column of cells is substantially similar to the width of a protrusion of a magnetic field modulator placed in physical contact with the surface. In other cases, a magnetic field modulator positioned proximate the surface, but not in physical contact with the surface (e.g., such that a gap is formed between the modulator and the surface), may cause weaker magnetic field intensities near the surface. This may occur since the magnetic field intensity decreases with increasing distance from the magnetic field source. In some embodiments, a magnetic field modulator positioned away from the surface can allow the formation of relatively narrow patterns of cells proximate a surface. For instance, the width of a column of cells may be substantially narrower than the width of a protrusion of the magnetic field modulator.

### EXAMPLE 9

Patterning of Multiple Cell Types Proximate a Surface

In some embodiments, magnetic field modulators can be used to form patterns of more than one type of cell proximate a surface. Culturing of multiple cell types, especially in controlled spatial arrangements proximate a surface, has a wide variety of applications. For instance, such arrangements of cells can enable study of molecular interactions between different types of cells and the formation of tissues comprising multiple cell types. Since tissues of some organisms exhibit a distinct micro-architecture defined by the spatial relationship between different cell types, patterning cells in controlled spatial arrangements can allow the study of the functional significance of such architectures. In some instances, the patterning of multiple cell types, especially in three-dimensional configurations, can lead to the formation of bioartificial organs.

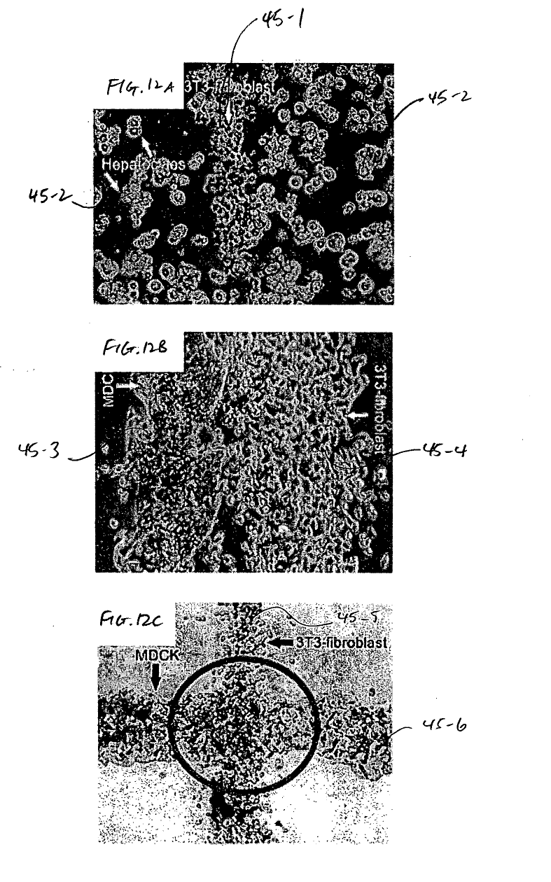

In one embodiment, a first set of cells can be patterned proximate a surface using a magnetic field modulator and a second set of cells can be deposited on the same surface without using a magnetic field modulator to position the second set of cells. For example, FIG. 12A shows a first set of cells 45-1 (fibroblasts) patterned in the form of a column on a surface, and a second set of cells 45-2 (hepatocytes) positioned randomly on the surface. This method shows that active attraction of a first set of cells (e.g., attraction of magnetically-loaded fibroblasts to a magnetic field modulator) can be combined with passive attraction of a second set of cells (e.g., general attraction of cells to a collagen-coated surface) on a single surface.

In some embodiments, multiple types of cells can be positioned proximate one another on a surface. In one particular embodiment, multiple types of cells can be positioned beside one another on a surface. For instance, as illustrated above in FIGS. 1F and 1G, a first set of cells can form a pattern proximate a surface using a magnetic field modulator placed at a first position, and the modulator can be moved in relation to the surface to a second position. (I.e., either the modulator or the surface can be moved, as long as one is moved in relation to the other). While the modulator is in the second position, a second set of cells can be patterned proximate the surface. For example, in one particular embodiment, a first set of cells 45-3 (MDCK cells) are positioned proximate a surface, and after moving the magnetic field modulator 200 μm adjacent the first position, a second type of cells (3T3fibroblast) are positioned proximate the first set of cells (FIG. 12B). Thus, a co-culture system of cells can be formed proximate the surface.

### EXAMPLE 10

Positioning of Multiple Cell Types Proximate a Surface

In yet another embodiment, multiple types of cells can be positioned on top of one another on a surface. For example, as illustrated in FIGS. 1D and 1E, multiple types of cells can be patterned on top of one another without moving the magnetic field modulator in relation to the surface. In other cases, multiple types of cells can be positioned on top of one another by first patterning a first set of cells while a magnetic field modulator is in a first position, and then patterning a second set of cells while the magnetic field modulator is in a second position. For example, as shown in FIG. 12C, a first set of cells 45-5 (e.g., fibroblasts) can be positioned proximate a surface. After the attachment of cells 45-5 to the surface and the formation of a first pattern of cells, the magnetic field modulator can be turned at an angle (e.g., 90 degrees) in relation to the first pattern of cells. A second set of cells 45-6 (e.g., MDCK cells) can be patterned on top of the first set of cells. The circle in FIG. 12C indicates a defined area where the cells overlay. In some instances, the cells in this area can form a three-dimensional cellular structure, as described in more detail below. Of course, additional sets (e.g., third, fourth, and fifth sets) of cells can also be patterned on top of one another if desired.

As demonstrated herein, a first pattern of cells can be positioned proximate a second pattern of cells with micron precision. In some instances, at least one feature of a first pattern of cells may be separated by a certain distance from at least one feature of the second pattern of cells. For example, one or more features of a first pattern of cells may be separated by less than 500 microns, less than 300 less than, less than 200 microns, less than 100 microns, or less than 50 microns from one or more features of the second pattern of cells.

Any of a variety of cells may be patterned using methods and apparatus of the present invention (e.g., mammalian, bacterial, and plant cells). In some embodiments, a first and a second set of cells are the same. In other embodiments, a first and a second set of cells have at least one different characteristic. For example, the first and second set of cells may be different cell types, or they may be the same cell type but have other differing characteristics (i.e., internal and/or external to the cell) such as protein expressions.

In one embodiment, a pattern comprising an array of cells in defined spatial arrangements is formed on a surface. In some cases, a first set of cells in a defined spatial arrangement may have a different characteristic from a second set of cells in a defined spatial arrangement on the surface. In one particular embodiment, the first set of cells may be of one cell type and the second set of cells may be of a different cell type. Of course, third, fourth, fifth, etc. sets of cells of the same or different cell types can also be patterned on the surface. Such a cellular array has many potential uses, i.e., for forming micro-tissues comprising different cell types, for studying cell-cell interactions, and/or for performing cellular assays.

As shown above, some embodiments of the invention can be used to pattern cells in controlled spatial arrangements in-two dimensions. In certain embodiments, these methods can be further extended to pattern cells in three-dimensions. “Three-dimensional cellular structures,” as used herein, means that the smallest dimension of the cellular structure (e.g., length, width, depth, or cross-sectional dimension) is a least 100 microns. A cross-section dimension may include, for instance, the distance between two opposed points of a surface, or surfaces, of a hollow or partially hollow structure comprising cells, such as a semi-tubular or tubular structure. In some cases, three-dimensional patterning of cells may be carried out by attracting cells to inner and/or outer surfaces of three-dimensional structures that can be used as templates for cells. These three-dimensional templates may include, for instance, single tubules or complex tubular structures, such as branching structures. Mono-layers or multi-layers of cells can be patterned on such structures. Three-dimensional patterning of cells can be useful for tissue engineering, e.g., for forming three-dimensional cellular structures such as tissues and organs (or parts thereof), including kidney, lung, liver, and vessels.

### EXAMPLE 11

Patterning of Cells in Three Dimensions

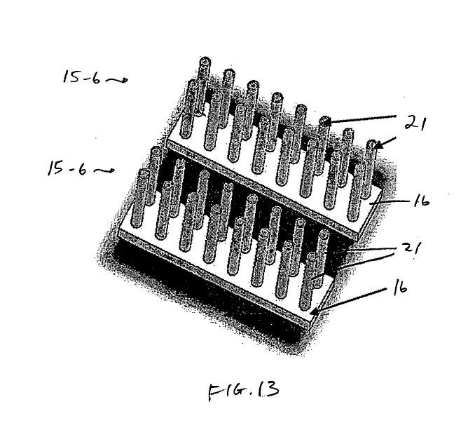

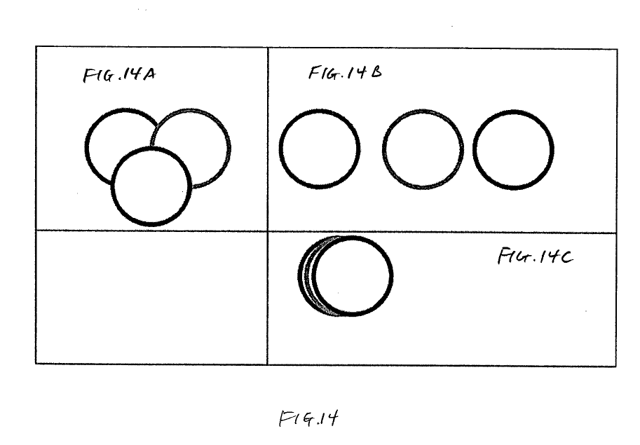

FIG. 13 shows an example of a “ring field modulator”, magnetic field modulator 15-6, including base 16 and protrusions 21. Magnetic field modulator 15-6 may be used to pattern cells in circular or ring-shaped patterns, and, in some cases, to pattern cells into three-dimensional cellular structures. Protrusions 21 can be formed from, for instance, hollow steel pins having a 200 μm internal diameter. Of course, other dimensions, configurations, or suitable materials can be used to fabricate the modulator or portions thereof. Modulator 15-6 can be used to form different patterns of cells. For instance, as shown in FIG: 14A, cells-can be positioned by modulators as partially overlaying cell circles. Cell circles positioned as non-overlapping circles can also be formed (FIG. 14B). Both of these approaches can enable the study of controlled cell-cell interactions in defined areas that can be analyzed, i.e., by optical detection. In some instances, magnetic field modulator 15-6 can be used to attract cells in a tubular fashion. For instance, as shown in FIG. 14C, cell circles can be position as multilayered structures on top of one another to form three-dimensional cellular structures such as micro-tissues. In one embodiment, a three-dimensional cellular structure may be formed by adding cells to a surface layer by layer. In another embodiment, a three-dimensional cellular structure comprising architectures of both macro and micro dimensions can be assembled. A three-dimensional cellular structure may comprise tissue-specific micro-architectures, for example, renal tubules surrounded by small capillaries.

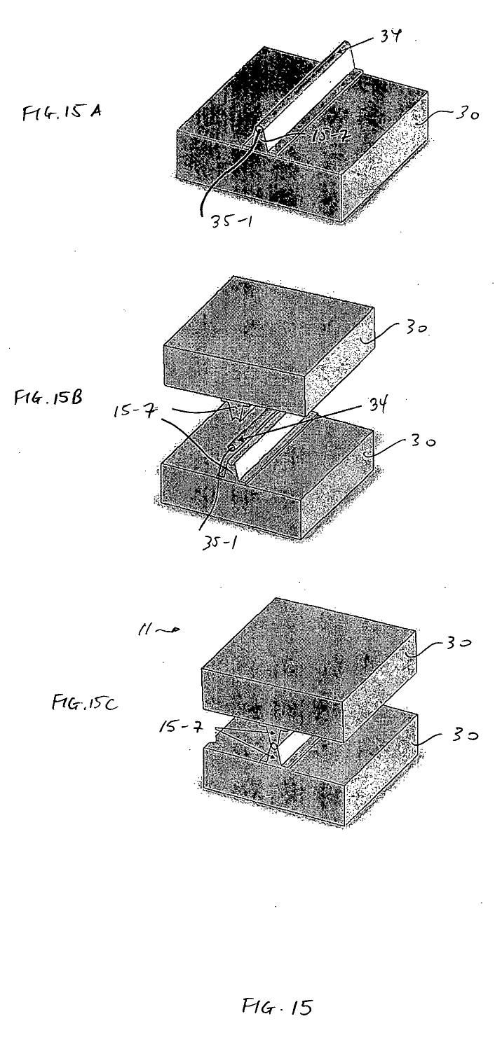

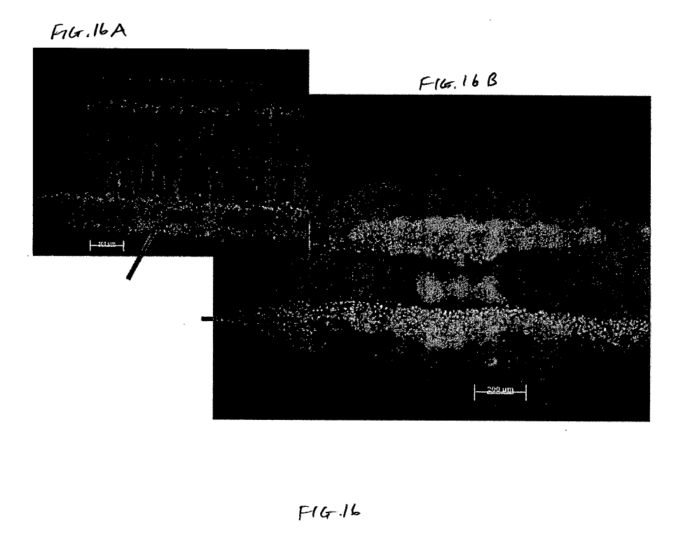

In some embodiments, cells can be patterned into a three-dimensional tubular structure. Such a structure can be formed, for instance, using the embodiments illustrated in FIG. 15. FIG. 15 shows an example of a “semi-tubular field modulator”, magnetic field modulator 15-7, having a concave shape and a configuration complementary to that of tubular structure 34. As shown in FIG. 15A, tubular structure 34 can be positioned proximate magnetic field modulator 15-7. In some cases, multiple magnetic field modulators 15-7 can be used (FIG. 15B). Permanent magnets may serve as magnetic field sources 30, and can be positioned proximate the magnetic field modulators. In certain embodiments, inner surface 35-1 of tubular structure 34 can be used as a surface for patterning cells into-a three-dimensional tubular structure. For example, a fluid containing a plurality of cells can be deposited inside tubular structure 34, and cells may attract surface 35-1 due to the magnetic field gradients produced by magnetic field modulators 15-7. FIGS. 16A and 16B are photographs showing renal cells patterned in tubular structure 34 after 12 hours. In some cases, the amount of time required to culture cells into tubular structures using methods of the present invention is significantly shorter than certain conventional methods, which may take up to 10 days. Therefore, the methods described herein may be useful for accelerating the cell seeding process and/or the preparation of bioartificial devices.

### OTHER EMBODIMENTS

Magnetic field modulators may be capable of attracting, trapping, and/or controlling the location of cells associated with magnetic materials. As such, a magnetic field modulator can be used to form a variety of patterns of cells proximate a surface. A pattern of cells can comprise one or a plurality of the same or different features. For instance, a pattern may comprise features in the shapes of squares, circles, ovals, lines, and the like. In some embodiments, the features are areas where cells are positioned (e.g., if the feature is a square, cells may be positioned in the shape of the square). In other embodiments, the features are areas where cells are absent (e.g., if the feature is a square, cells may be positioned around the perimeter of the square, but not in the square). The features may be interconnected, or discrete in some cases. Patterns of cells proximate a surface may be controlled by the shape, configuration, and dimensions of the magnetic field modulator, the material in-which the magnetic field modulator is made, and the position of the magnetic field modulator in relation to the surface. In some cases, discrete patterns of cells in two and/or three-dimensional arrays can be formed, i.e., the magnetic field modulator may be used to attract cells in certain discrete areas proximate a surface. In other cases, the magnetic field modulator may be used to pattern cells by concentrating the cells in a general area proximate a surface and/or to form a high density of cells proximate a surface. Attracting cells towards the inner perimeter of a tube-like surface, as shown in FIG. 16, is one example of such a method.

As described above, the shape of a magnetic field modulator can vary. For example, a magnetic field modulator may include one or a plurality of protrusions (e.g., as shown in FIG. 2), or a concave area that may facilitate immobilization of cells (e.g., as shown in FIG. 15). In some cases, e.g., as shown in FIG. 13, a modulator can have protrusions that are supported by a base. In other cases, modulation of a magnetic field can be achieved using a series of “protrusions” that are not supported by a base, i.e., each protrusion may act as a magnetic field modulator. In yet other cases, a magnetic field modulator can be substantially flat. Of course, other shapes and/or configurations of magnetic field modulators are possible.

Magnetic field modulators can be fabricated with features having a range of dimensions (e.g., from centimeters to micrometers). For instance, a magnetic field modulator may have at least one feature having a dimension of less than 1 cm, less than 1 mm, less than 500 microns, less than 300 microns, less than 200 microns, or less than 100 microns, less than 50 microns, or less than 10 microns. In some cases, the pattern of cells formed on a surface has at least one feature with a dimension on the order of a dimension of the magnetic field modulator. For example, a magnetic field modulator having a protrusion with dimensions 200 μm by 200 μm (e.g., in the shape of a square) may be used to form a pattern of cells in the shape of a ˜200 μm by 200 μm square proximate a surface. In another embodiment, a magnetic field modulator having a protrusion with dimensions 10 μm by 10 μm may be used to form a pattern of cells in the shape of a ˜10 μm by 10 μm square proximate a surface. Of course, the dimensions and shape of the pattern of cells can be regulated by other factors or methods such as varying the distance between the magnetic field modulator and the surface, as discussed above.

Magnetic field modulators can be made in any suitable material that can be used to modulate a magnetic field. In some embodiments, magnetic field modulators are formed from ferromagnetic materials. Ferromagnetic materials, such as iron (Fe), nickel (Ni), cobalt (Co), gadolinium (Gd), and various alloys, are materials that can be easily magnetized. In other words, ferromagnetic materials can exert an attractive or repulsive force on other materials. Sometimes, magnetic field modulators are made from steel (a ferromagnetic material). Steel is a generally hard, strong, durable, malleable alloy of iron and carbon, usually containing between 0.2 and 1.5 percent carbon, often with other constituents such as manganese (Mn), chromium (Cr), nickel (Ni), molybdenum (Mo), copper (Cu), tungsten (W), cobalt (Co), or silicon (Si), depending on the desired alloy properties. In one particular embodiment, a magnetic field modulator was made from a low carbon steel, e.g., Steel 1010. Other non-limiting examples of ferromagnetic materials that can form magnetic field modulators include FeOFe₂O₃, NiOFe₂O₃, CuOFe₂O₃, MgOFe₂O₃, MnBi, MnSb, MnOFe₂O₃, Y₃Fe₅O₁₂, CrO₂, MnAs, Dy, and EuO.

In some embodiments, magnetic field modulators are formed from paramagnetic materials. Paramagnetic materials attract and repel like normal magnets when subject to a magnetic field, i.e., the dipoles of the material align with an external magnetic field. Alignment does not occur, however, when the material not subjected to a magnetic field. Non-limiting examples of paramagnetic materials include aluminum (Al), barium (Ba), calcium (Ca), oxygen (0), platinum (Pt), sodium (Na), strontium (Sr), uranium (U), magnesium (Mg), and technetium (Tc).

Techniques for fabricating magnetic field modulators can vary depending on the desired shape of the magnetic field modulator, the type of material in which the modulator is made, etc. Those of ordinary skill in the art can determine appropriate techniques and materials used to fabricate magnetic field modulators. Techniques such as wire cutting processes (e.g., wire-cut machining processes using micron-sized wires) and photolithography can be used to fabricated modulators in some cases. In other cases, commercially available materials (e.g., steel tubes) can be used as magnetic field modulators. In some embodiments, materials suitable for forming magnetic field modulators (e.g., ferromagnetic particles) can be shaped by embedding them into polymers.

In some embodiments, the magnetic field modulator is not integrally connected to a surface for directing formation of patterns of cells. As used herein, the term “integrally connected,” when referring to two or more objects, means objects that do not become separated from each other during the course of normal use, and/or separation requires causing damage to at least one of the components, for example, by breaking, peeling, etc. (separating components fastened together via adhesives, tools, etc.). For instance, in one embodiment, the magnetic field modulator is not embedded into a surface (i.e., either partially or completely). Advantageously, a non-integrally connected magnetic field modulator cam allow the modulator to be repositioned without altering the surface. For instance, in some cases, the surface does not have to be manipulated or moved in order to reposition the magnetic field modulator. This can allow, for instance, the patterning of a second set of cells on the same surface and/or the patterning of a second set of cells on a different surface using the same modulator.

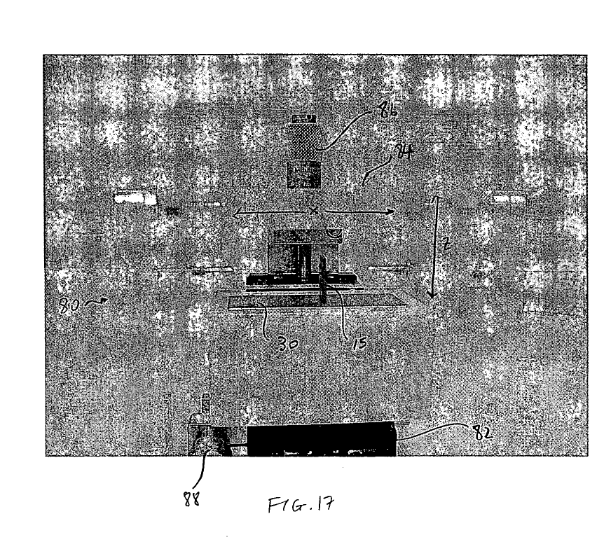

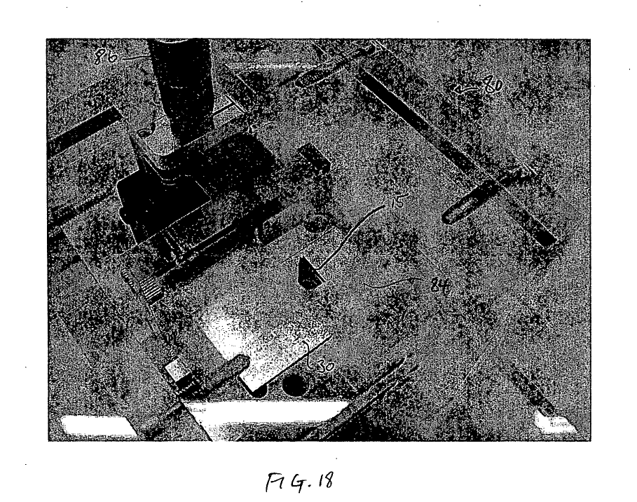

In some embodiments, positioning of a magnetic field modulator proximate a surface can be facilitated by the use of a stage apparatus. An example of such an apparatus is shown in FIGS. 17 and 18. As shown in the embodiment illustrated in FIG. 17, stage apparatus 80 includes platform 82 for supporting magnetic field source 30 and magnetic field modulator 15. A surface (e.g., a petri dish or glass slide) for directing formation of cell patterns can be positioned on supporting member 84. Magnetic field modulator 15 can be positioned proximate supporting member 84 (and can therefore be positioned proximate a surface for patterning cells) using vertical (z) control 86 and horizontal (x) control 88. These controls can allow movement of the magnetic field modulator with micrometer precision. It should be understood that the use of a stage apparatus is by way of example only, and those of ordinary skill in the art will know of additional techniques suitable for positioning magnetic field modulators proximate a surface.

Certain embodiments of the invention involve contacting a surface with a fluid containing a plurality of cells. In some cases, a fluid containing a plurality of cells can be deposited on a surface using a pipette (e.g., a manual or automated pipette) or another dispensing device. In other cases, fluids containing cells can be deposited on a surface by flowing the fluid on a surface. For instance, a surface may be in contact with, and/or a part of, a fluidic apparatus (e.g., a microfluidic device). The fluid can be flowed through a channel of the apparatus and cells in the fluid may be attracted to a magnetic field modulator positioned proximate the surface while the fluid is flowing or while the fluid is stationary.

A variety of different surfaces can be used for directing formation of patterns of cells, especially for embodiments in which the magnetic field manipulator is not integrally-connected to the surface. Any suitable material can be used to fabricate surfaces, including polymers (e.g., polystyrene) and non-polymers (e.g., glass, quartz, ceramics, and silicon). Surfaces may be biodegradable or non-biodegradable. In some cases, surfaces are flexible (i.e., elastic) and can be configured to have different shapes. Surfaces may have any suitable shape and may be curved, tube-like, substantially planar, rough, smooth, porous, and/or patterned with features (e.g., micron-sized features). In some embodiments, surfaces may be sterile and may contain layer(s) of proteins (e.g., extracellular matrix proteins) to facilitate cell attachment. Surfaces may also include those of scaffolds or other materials for implanting into a mammalian body and/or surfaces that facilitate the fabrication of bio-artificial organs.

In certain embodiments of the invention, cells are associated with a magnetic material. Cells can be associated with a magnetic material, e.g., by loading (i.e., tagging) the cells with magnetic materials such as magnetic beads or particles. The magnetic material may vary in size but are generally less than 1 μm in diameter. For instance, the magnetic material may be less than 0.5 μm in diameter, less than 0.1 μm in diameter, or less than 10 nm in diameter. Magnetic materials may be formed in any suitable material for interacting with a magnetic field. Examples include ferromagnetic and superparamagnetic materials.

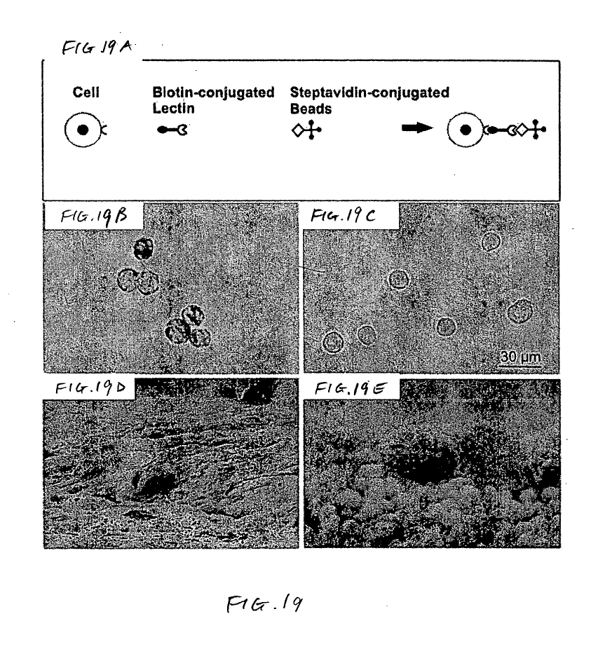

In some instances, cells can-be associated with magnetic materials through antibodies conjugated to the magnetic material, which bind to specific cell membrane receptors. In one particular example, cells can be loaded with streptavidin-conjugated beads (e.g., Dynabeads) by subjecting the cells to a solution containing the streptavidin-conjugated beads and a solution containing biotin-conjugated lectin (FIG. 19A). Other examples include anti-Ig kappa light chain antibody, anti-CD45R antibody, or anti-syndican, which can be used to bind to activated B-cells. The amount of antibody required for binding a magnetic material to a cell will depend on the antibody affinity as well as antigen density of the target cell population. Such antibody-coated magnetic beads may be phagocytosed by the cells to which they bind. Alternatively, in some embodiments, a magnetic material itself may function as a bioaffinity ligand. For instance, particles of Fe₂FO₃ can adhere to the cell surfaces of certain cells such as Saccharomyces cerevisiae; making the cells magnetic. A variety of different techniques for associating cells with magnetic materials are known or may be determined by those of ordinary skill in the art.

In certain cases, cells are naturally associated with a magnetic material. For instance, magnetic bacteria such as those of the Geobacter family (e.g., G. metalloreducens), consume rust (Fe(OH)₂) and reduce the rust to magnetite (Fe₃O₄), a naturally magnetic material. The magnetite within the bacteria enable the bacteria to move in a directed fashion along magnetic field lines.

The following examples are intended to illustrate certain embodiments of the present invention, but are not to be construed as limiting and do not exemplify the full scope of the invention.

### EXAMPLE 12

Procedures for Culturing Cells

This example shows procedures for culturing cells for cell patterning according to certain embodiments of the invention.

Madin-Darby Canine Kidney cells (MDCK), C2C12 muscle cells, NIH 3T3 fibroblast cells and glioblastoma cell line U-87 were obtained from American Type Culture Collection (Rockville, USA). All four cell types were cultured in DMEM (Invitrogen Singapore Pte Ltd, Singapore) with 10% fetal calf serum (Invitrogen Singapore Pte Ltd, Singapore) and 1% antibiotic-antimycotic solution (Invitrogen Singapore Pte Ltd, Singapore). Primary hepatocytes were isolated from wistar rats by a two-step in-situ collagenase digestion (Seglen, 1976). During all the procedures, the primary hepatocytes were cultured in Hepatozym-serum free culture medium (Invitrogen Singapore Pte Ltd, Singapore) with 10⁻⁷ dexamethasone (Sigma-Aldrich, Singapore) and 1% antibiotic-antimycotic solution (Invitrogen Singapore Pte Ltd, Singapore) was used.

### EXAMPLE 13

Procedures for Associating Cells with Magnetic Materials

This example shows procedures for loading cells with magnetic materials for cell patterning according to certain embodiments of the invention.

Dynabeads MyOne Srepatavidin C1 (Innovative Biotech, Singapore) were used as magnetic materials for associating with cells and were prepared according to the manufacture's instructions, as illustrated generally in FIG. 19A. A 100 μL volume of the bead solution was added to 500 mL of sterile PBS+0.1% BSA+0.01% Tween-20 containing 25 μL of biotinylated wheat germ agglutinin (Vector Laboratories, Burlingame, USA), and the resulting suspension was mixed for 30 minutes at room temperature using a Dynal sample mixer (Innovative Biotech, Singapore). A 20 μL volume of the resulting lectin-bead solution was added to approximately 7.5 million trypsinized cells and gently incubated for 15 minutes at room temperature using a Dynal sample mixer. FIG. 19B shows cells in suspension after exposure to lectin and beads; beads were bound to the cells. Cells in suspension that were exposed to the beads only, without lectin, lead to cells without attached beads. This observation indicates the specificity of the binding process caused by the lectin (FIG. 19C).

Alternatively, 10 μL of the lectin-bead solution can be added to a confluent layer of cells in a 75 cm² culture flask and incubated under gently shaking conditions under 5% CO₂ and 37° for 30 minutes. FIGS. 19D and 19E show beads loaded on a monolayer of cells. Subsequently, the cells were trypsinized. The bead-loaded cells were then separated from the non-bead loaded cells in a MCP-S magnetic stand (Innovative Biotech, Singapore) and finally counted using a Neubauer-improved counting chamber (Paul Marienfeld, Germany). Both procedures led to a loading of 10 to 30 beads per cell.

This example demonstrates that cells can be tagged with magnetic particles using simple procedures according to embodiments of the invention.

### EXAMPLE 14

Procedures for Fabricating Magnetic Field Modulators

This example shows a procedure for fabricating a magnetic field modulator according to certain embodiments of the invention.

In some embodiments, magnetic field modulators, such as modulators 15 as shown in FIG. 17 and modulators 15-2 to 15-5 as illustrated in FIG. 6, were made from low carbon steel. The modulators can be fabricated by electrical discharge machining (EDM), a precision wire-cut machining process that uses a metal wire electrode to cut a programmed contour in a block (i.e., a precursor structure). The magnetic field modulators were machined from a block of low carbon steel based on engineering drawing and dimensions. The wire was made from a brass alloy and can range from about 20-330 μm in diameter. In order to cut conical blocks or different profiles on the top and bottom of a block, the wire was angled relative to the block. The resolution of this machining was dependent on the size of the wire, which was equal to or greater than 20 microns. EDM can be a useful technique for fabricating modulators when the modulator is made in a hard material, and/or when the complexity of portions of the modulator (e.g., features having high aspect ratios) makes conventional fabrication techniques difficult or impossible.

In another embodiment, portions of the modulator may be assembled i.e., onto a base. For instance, magnetic field modulator 15-6, as shown in FIG. 13, was made using hollow steel pins with internal diameters of 200 μm, which served as protrusions 21. Base 16 of the magnetic field modulator was made out of low carbon steel, with an array of 0.5 mm through holes. The steel pins were then mechanically inserted into these holes with a tight fit to ensure that the pins did not loosen and/or fall through the holes. The base was then grounded to ensure that a flat surface was achieved.

In other embodiments, magnetic field modulators can be fabricating using lithography, e.g., optical lithography and electron-beam lithography. Modulators made by such techniques may include, for instance, a silicon substrate having conductive layers. Conductive layers can be formed by depositing metal films (e.g., gold, silver, titanium, and/or platinum) on the substrate by methods such as electroplating, vacuum deposition, and thermal evaporation. The conductive layers can act as conducting wires, which can be fabricated to have dimensions of less than 10 microns. In some cases, the electromagnetic fields produced by the conducting wires can be modulated by controlling the current density through the wires. A modulator comprising an exposed and developed photoresist pattern on silicon can be prepared using techniques described in any conventional lithography text, such as Introduction to Microelectronic Fabrication, by Richard C. Jaeger, Gerold W. Neudeck and Robert F. Pierret, eds., Addison-Wesley, 1989.

This example demonstrates that magnetic field modulators can be made using a variety of different materials and methods, according to certain embodiments of the invention.

### EXAMPLE 15

Procedures for Patterning Cells Proximate a Surface

This example shows that patterns of cells can be formed proximate a surface using a magnetic field modulator according to certain embodiments of the invention.

Culture dishes (e.g., petri dishes) and cover slides were used as received or were coated with collagen and used as surfaces for patterning cells. Magnetic field modulators were fabricated using methods described in Example 3. A surface was placed on a stage apparatus (e.g., on supporting member 84 of stage apparatus 80, as shown in FIG. 17) and the stage apparatus was used to move the magnetic field modulator toward the surface (i.e., in the vertical z-direction). A 1 mL volume of culture medium was added to the surface on an area above the field modulator. Subsequently, 20,000 to 60,000 cells, which were tagged with Dynabeads using the procedure described in Example 2, were added to the 1 ml of culture medium using a 1000-μL pipette, and the complete set-up was transferred to a CO₂ incubator. Within minutes, fine lines of cells could be observed congregating above the protrusions of the magnetic field modulator. After 4-5 hours of incubation, the magnetic field modulator was removed and non-adhered cells on the substrate were gently washed away with PBS. The surface with the adhered cells was cultured further on to form confluent columns of cells. An Olympus CKX 41 microscope (Olympus, Singapore) was used to visualize the cell patterns. Images were taken with a digital camera and thereafter processed with Photoshop 5.5 (Adobe Systems, San Jose, Calif.).

This example demonstrates that a magnetic field modulator can be used to align cells and to form patterns of cells proximate a surface. This example also shows that cells can form patterns on different types of surfaces (e.g., surfaces of petri dishes and glass slides); in other words, cells can be patterned independently of the surface on which the cells adhere.

### EXAMPLE 16

Procedures for Forming Multiple Patterns of Cells Proximate a Surface

This example shows that patterns of different cell types can be formed proximate a surface using a magnetic field modulator according to certain embodiments of the invention.

A procedure similar to the one described in Example 4 was used to form a first pattern of cells at a first position proximate a surface. The first pattern of cells included cells aligned in columns that were approximately 200 μm wide, separated from the adjacent columns by an approximately 200 μm wide spacing. To form a co-culture of cells, the magnetic field modulator was moved from its first position to a second position 200 μm away (horizontally in the x-direction) from the first position using a movable micro-stage, i.e., the protrusions of the magnetic field modulator were positioned underneath the 200 μm wide spacings of the first pattern of cells. The second position was determined by fine adjustments of the horizontal stage. Using a cell culturing technique similar to that described in Example 4, a second set of cells was patterned proximate the surface while the magnetic field modulator was in the second position. The second set of cells, which were also tagged with magnetic particles, aligned with the protrusions of the magnetic field modulator in the second position. This procedure led to the patterning of a co-culture of cells proximate the surface. An Olympus CKX 41 microscope (Olympus, Singapore) was used to visualize the cell patterns. Images were taken with a digital camera and thereafter processed with Photoshop 5.5 (Adobe Systems, San Jose, Calif.).

This example demonstrates that multiple types of cells can be patterned on a single surface by repositioning a magnetic field modulator in relation to the surface. This example also shows that patterning of multiple cell types is independent of the surface on which the cells are patterned.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of forming a pattern of cells proximate a surface, comprising:
providing a surface for directing formation of a pattern of cells;
providing a magnetic field;
positioning a magnetic field modulator in a first position in relation to the surface and modulating the magnetic field, wherein the magnetic field modulator is not integrally connected to the surface;
contacting the surface with a fluid containing a first set of cells; and
forming a first pattern of cells proximate the surface, wherein the position of the first pattern of cells proximate the surface is determined at least partially by the first position of the magnetic field modulator.

2. A method as in claim 1, further comprising contacting the surface with a fluid containing a second set of cells, and forming a second pattern of at least a portion of the second set of cells on the surface.

3. A method as in claim 2, wherein at least a portion of the second pattern of cells is positioned on top of at least a portion of the first pattern of cells.

4. A method as in claim 2, wherein at least one feature of the first pattern of cells is separated by less than 500 microns from at least one feature of the second pattern of cells.

5. A method as in claim 2, wherein the first and second sets of cells have at least one different characteristic.

6. A method as in claim 5, wherein the first and second sets of cells are different cell types.

7. A method as in claim 5, wherein the first and second sets of cells are the same cell types.

8. A method as in claim 1, further comprising attaching the patterned cells onto the surface.

9. A method as in claim 1, wherein contacting the surface with a fluid comprises flowing the fluid across the surface.

10. A method as in claim 1, wherein the cells form a three-dimensional cellular structure.

11. A method as in claim 10, wherein the three-dimensional cellular structure comprises a tissue.

12. A method as in claim 11, wherein the tissue comprises more than one type of cell.

13. A method as in claim 1, wherein the first pattern of cells comprises at least one feature with a dimension of less than 300 microns.

14. A method as in claim 1, wherein the first pattern of cells comprises at least one feature with a dimension of less than 100 microns.

15. A method as in claim 1, wherein the surface is flexible.

16. A method as in claim 1, wherein the surface is tubular or semi-tubular.

17. A method of forming patterns of cells proximate a surface, comprising:
providing a surface for directing formation of patterns of cells;
providing a magnetic field;
positioning a magnetic field modulator in a first position in relation to the surface and modulating the magnetic field;
forming a first pattern of a first set of cells proximate the surface, wherein the position of the first pattern proximate the surface is determined at least partially by the first position of the magnetic field modulator;
positioning the magnetic field modulator in a second position in relation to the surface and modulating the magnetic field, wherein the first and second positions are different; and
forming a second pattern of a second set of cells proximate the first pattern of the first set of cells, wherein the position of the second pattern is determined at least partially by the second position of the magnetic field modulator.

18. A method as in claim 17, wherein the magnetic field modulator is not integrally connected to the surface.

19. A method of forming a pattern of cells proximate a surface, comprising:
providing a surface for directing formation of a pattern of cells;
providing a magnetic field;
positioning a magnetic field modulator in a first position relative to the surface and modulating the magnetic field;
contacting the surface with a fluid containing a first set of cells;
forming a first pattern of cells proximate the surface, wherein the position of the first pattern is determined at least partially by the first position of the magnetic field modulator; and
causing the cells to form a three-dimensional cellular structure.

20. A method as in claim 19, wherein the magnetic field modulator is not integrally connected to the surface.

21. A method as in claim 19, wherein the three-dimensional cellular structure comprises a tissue.

22. An apparatus for forming a pattern of cells proximate a surface, comprising:
a surface for forming a pattern of cells;
a magnetic field; and
a magnetic field modulator positioned adjacent the magnetic field and the surface, wherein the magnetic field modulator is not integrally connected to the surface.
